(19) United States
(12) Patent Application Publication
(10) Pub. No.: US 2004/0093079 A1
(43) Pub. Date: May 13, 2004

(54) Compositions and methods for the reduction of post-angioplasty stenosis

(76) Inventors: David A. Tulis, Raleigh, NC (US); William Durante, Houston, TX (US); Andrew I. Schafer, Philadelphia, PA (US)

Correspondence Address: Tim Headley, GARDERE WYNNE SEWELL LLP, Suite 3400, 1000 Louisiana, Houston, TX, 77002-5007, US

(21) Appl. No.: 10/307,898

(22) Filed: Dec. 2, 2002

Related U.S. Application Data
(60) Provisional application No. 60/334,454, filed on Nov. 30, 2001.

(51) Int. Cl. A61F 2/06

(52) U.S. Cl. 623/1.42

(57) ABSTRACT

The present invention includes compositions and methods for administering YC-1 to reduce post-angioplasty restenosis. The compositions include stents and catheters coated with YC-1 and kits with YC-1 coated catheters or stents.

The present invention was supported, in part, by the National Heart, Lung, and Blood Institute grants BL-36045, HL-59976, and HL-62467, and the Veterans Affairs Merit Review Board. The federal government may have certain rights in the invention.

### TECHNICAL FIELD OF THE INVENTION

The present invention relates generally to cardiology and, in particular, to compositions and methods for reducing post-angioplasty stenosis and for mitigating vascular injury caused by angioplasty or embolectomy.

This application claims priority from U.S. Provisional Application No. 60/334,454 filed on Nov. 30, 2001.

### BACKGROUND OF THE INVENTION

The pathophysiologic process of blood vessel stenosis following invasive balloon catheterization continues to be an enigmatic latrogenic complication in the clinical setting. Arterial remodeling subsequent to endovascular balloon angioplasty manifests as a pathologic and proliferative neointima with a major diminution in luminal patency. Clinical symptoms of the arterial remodeling events include angina, shortness of breath, claudicatiori, radiating nerve sensation, and other cardiovascular-related phenomena that may result in stroke and/or myocardial or systemic infarct. Although, in the acute setting, the success rate of balloon angioplasty may be greater than 95%, long-term success continues to be limited by the eventual restenotic narrowing of the treated vessel. Vessel restenosis currently occurs in 30-40% of patients within 6 months after initial balloon catheterization (Lincoln, T. M., Dey, N. B., Boerth, N. J., Cornwall, T. L., and Soff, G. A., Nitric oxide-cyclic GMP pathway regulates vascular smooth muscle cell phenotype modulation: implications in vascular disease; Acta. Physiol. Scand., 164: 507-515, 1998; Kaul, S., Cercek, B., Rengstrom, J., Xu, X-P., Molloy, M. D., Dimayuga, P., Pan'kh, A. K., Fishbein, M. C., Nilsson, J., Rajavashisth, T. B., and Shah, P. K., Polymeric-based perivascular delivery of a nitric oxide donor inhibits intimal thickening after balloon denudation arterial injury: role of nuclear factor-kappaB; J Am. Coll. Cardiol., 35: 493-501, 2000; Wagner, C. T., Durante, W., Christodoulides, N., Hellums, J. D., and Schafer, A. I., Hemodynmic forces reduce the expression of heme oxygenase in cultured vascular smooth muscle cells. J Clin. Invest., 100: 589-596, 1997).

With restenosis, subsequent decisions must be made as to whether to attempt recanalization of the vessel with subsequent angioplasty, or choose alternative methods such as coronary artery bypass grafting (CABG) or vascular stent deployment. According to the U.S. Multicenter stent registry, 54% of patients receiving repeat balloon angioplasty also have recurrence of stenosis (Kozma, F., Johnson, R. A., Zhang, F., Yu, C., Tong, X., and Nasjletti, A., Contribution of endogenous carbon monoxide to regulation of diameter in resistance vessels, Am. J Physiol., 276: R1087R1094, 1999). In 1999, it was estimated that more than 1.3 million initial and repeat angioplasty procedures were performed worldwide Unfortunately, more than 2,600 people in the United States alone die per day from this cardiovascular disease complications such as from antioplasties, meaning that there remains a large need for appropriate methods and treatments of these persons.

For patients who have vascular disease, recanalization procedures are emotionally and physically disturbing as well as costly. Economic data analyses have determined expenditures associated with clinical balloon angioplasty surgeries. In the randomized Emory Angioplasty versus Surgery Trial, average cumulative cost per patient for the initial catheterization was $27,793, with an estimate of $44,491 per patient for an 8-year follow-up including all hospital and additional revascularization procedures if needed (Christodoulides, N., Durante, W., Kroll, M. H., and Schafer, A. I., Vascular smooth muscle cell heme oxygenases generate guanylyl cyclase-stimulatory carbon monoxide, Circulation, 91: 2306-2309, 1995). Even with the advent of artificial stent deployment adjuvent with balloon angioplasty, the restenosis rate remains high.

Adjuvent therapies to balloon angioplasty that could significantly diminish post-interventional vessel wall remodeling and luminal stenoses are greatly needed but have not yet been adequately identified. Although many characteristics of the adaptive responses to endovascular intervention have been characterized, the exact mechanisms and mediators responsible for architectural remodeling of the vessel wall following vascular recanalization remain unknown.

Of particular interest are the vascular cell products nitric oxide (NO) and carbon monoxide (CO), that serve as output modulators of the critical vascular enzymes NO synthase (NOS) and heme oxygenase (HO), respectively. Both of these diatomic gases stimulate soluble guanylyl cyclase (sGC) and thereby raise intracellular levels of cyclic guanosine monophosphate (cGNP), cyclic nucleotide-mediated signaling pathways ultimately result in regulation of important metabolic and functional processes within the cardiovascular system.

A novel and potentially relevant mechanism that sensitizes the sGC/cGW machinery has recently been described and represents an important new clinically therapeutic strategy. YC-1[3-(5′-hydroxymethyl-2′-furyl)-1-benzyl indazole] is a chemically synthesized compound that was originally shown to directly stimulate rabbit platelet sGC activity and cGMP production and subsequently inhibit platelet aggregation and thrombosis. Other documented effects of YC-1 include inhibition of ATP release from activated platelets, inhibition of platelet phosphoinositide breakdown, and suppression of intracellular Ca²⁺ rise from pro-aggregatory agonists.

YC-1 was determined, however, not to affect cGNT-specific phosphodiesterases (PDEs). Results from subsequent studies have shown that YC-1 stimulates sGC and cGMP independent of NO. See, for example, Friebe, A., Mullershausen, F., Smolenski, A., Walter, U., Schultz, G., and Koesling, D., YC-1 Potentiates Nitric Oxide-and Carbon Monoxide-Induced Cyclic GMP Effects in Human Platelets, Mol. Pharmacol., 54: 962-967, 1998; Wohlfart, P., Malinski, T., Ruetten, H., Schindler, U., Linz, W., Schoenafinger, K., Strobel, H., and Weimer, G., Release of Nitric Oxide from Endothelial Cells Stimulated by YC-1, an Activator of Soluble Guanylyl Cyclase, Br. J. Pharmacol., 128: 1316-1322, 1999; Stone, J. R., and Marletta, M. A., Synergistic Activation of Soluble Guanylate Cyclase by YC-1 And Carbon Monoxide: Implications for the Role Of Cleavage of the Iron-Histidine Bond During Activation by Nitric Oxide, Chem. Biol. 1998; 5: 255-261. YC-1 has been found, nevertheless, to potentiate both NO and CO-induced cGMP effects in human platelets.

The inventors have found that YC-1 exerts these sGC-stimulatory effects in both vascular endothelial and smooth muscle cells. YC-1 has been considered to exert its actions by stabilizing the active configuration of sGC and by decreasing the dissociation rates of the gases from the activated enzyme. Through these mechanisms, YC-1 is capable of stimulating CO-induced sGC with a similar potency and to a similar specific activity as that attained from NO-stimulation, approximately 400-fold. In addition, inhibition of the cGMP-specific hydrolyzing PDE-5 from YC-1 administration has been shown to occur in platelets and in aortic extracts. This apparently redundant but potentiating ability of YC-1 to both activate sGC and to concurrently cause a persistent elevation of cGMP (through inhibition of cGMP breakdown) makes it a selectively potent and attractive exogenous activator of the sGC/cGMP machinery.

As presented herein, the present invention is a novel post-angioplasty tool for improving clinical outcomes (e.g., reducing morbidity and mortality) currently associated with the surgical procedure. By regulating the role of gases associated with post-angioplasty stenosis, YC-1 serves as a therapeutic compound for mediating post-angioplasty neointimal development under clinical settings in mammals.

### SUMMARY OF THE INVENTION

Generally, and in one form, the present invention describes YC-1 as an attractive therapeutic agent with redundant protective mechanisms that regulate endogenous cGMP metabolism and attenuate post-injury arterial remodeling. An advantage of the present invention is that compositions and methods reduce the use of stenosis, including restenosis, which is often required post-angioplasty and which further contributes to blood vessel injury.

The present invention describes a method for the delivery of YC-1 in a copolymer gel to the ipsilateral artery immediately following balloon injury. Briefly, at least about after performing balloon angioplasty, a therapeutically effective dose of at least about 25% solution of a copolymer gel plus YC-1 is applied to an exposed adventitial surface of an injured artery. Control animals received 200 μl empty gel with 50 μl DMSO on the injured artery. In one embodiment, perivascular application of YC-1 compound in a copolymer gel allows complete and rapid adsorption within at least about 5 days in addition to a robust and highly significant attenuation of neointima formation following angioplasty in vessels exposed to YC-1.

The present invention provides methods of treating a patient to mitigate blood vessel injury and to prevent or reduce the need for restenosis. Treatment with the invention inhibits neointima and the aterial remodeling that occurs after luminal insult to a blood vessel. The methods involve administering to the patient a composition comprising a pharmaceutically effective amount of a soluble guanyl cyclase activator, such as YC-1.

In another embodiment of the present invention, the addition of YC-1 acute leads to an upregulation of endogenous vascular cGMP that may be significant, and a robust and consistent attenuation of angioplasty-induced neointima development in treated arterial sections. Importantly, YC-1 is found to sensitize sGC/cGMP machinery through stereotactic stabilization of the active configuration of sGC with reduction of the dissociation rates of CO and NO from the activated enzyme and through inhibition of the cGMP-dependent PDE. Together, the inventors have found that this provides YC-1 with a novel therapeutic role to robustly manipulate endogenous sGC and cGMP levels. Consequently, the cyclic nucleotide upregulation modifies the outcomes of stenosis following balloon angioplasty as demonstrated by consistent and significant attenuation of neointinial development.

The present invention, therefore, identifies YC-1 as a novel and therapeutically vital potentiator of endogenous GC/cGMP processes involved in reducing vessel stenosis and neointimal hyperplasia following arterial balloon angioplasty.

In yet another embodiment, the present invention is a method of treating a patient to mitigate blood vessel injury caused by angioplasty. The method involves administering to the patient a composition comprising a pharmaceutically effective amount of a soluble guanyl cyclase activator, such as YC-1. The soluble guanyl cyclase activator may also be a naturally occurring substance that accomplishes the same physiological function of YC-1 of the present invention.

An alternative method of the invention is a method of treating arterial trauma by forming a therapeutic composition including a pharmaceutically effective amount of a soluble guanyl cyclase activator in or on a biologically acceptable carrier and administering the composition on or near the locus of trauma so that neointima formation on or near the locus of trauma is reduced. The soluble guanyl cyclase activator may be YC-1 and the carrier may be a catheter, such as a balloon angioplasty catheter or an embolectomy catheter. Additionally, the carrier may be a gel such as a copolymer gel.

In further embodiments, the catheter is coated with a pharmaceutically effective amount of the soluble guanyl cyclase activator. The soluble guanyl cyclase activator may be administered during withdrawal of the balloon angioplasty catheter. Another example provides a catheter fitted with an applicator for delivery of the soluble guanyl cyclase activator.

Still further embodiments of the present invention include a stent coated with a therapeutic amount of YC-1. The stent may be permanent or comprise biodegradable component materials such as poly(L-lactic acid) (PLA), poly(glycolic acid), poly(L-lactic-co-glycolic acid) (PGA), poly(e-caprolactone) (PLGA), polyortho esters, polyanhydrides or combinations thereof, as examples. The biodegradable materials may be layered. In addition, gradients of YCI may be applied (e.g., along the length or width of the stent).

Additional embodiments include a balloon angioplasty catheter coated with YC-1 or a catheter fitted with an applicator for applying YC-1 during withdrawal of the catheter.

Other embodiments include a kit with a catheter adapted for administration of YC-1 to a blood vessel, a pharmaceutically effective amount of YC-1 adapted for administration by the catheter and instructions for administering the YC-1 with the catheter. The kit may include instructions printed on paper. The instructions, however, may be on a videotape or CD-ROM that may include, for example analog or digital, respectively, video demonstrations instructing on the use of the kit.

An advantage of the present invention is that compositions and methods reduce the use of stenosis, including restenosis, which is often required post-angioplasty and which further contributes to blood vessel injury.

### BRIEF DESCRIPTION OF THE DRAWINGS

For a more complete understanding of the features and advantages of the present invention, reference is now made to the detailed description of the invention along with the accompanying FIGURES in which corresponding numerals in the different FIGURES refer to corresponding parts, if applicable, and in which:

FIG. 1 is schematic drawing depicting YC-1 administration on balloon injured left carotid artery;

FIG. 2 depicts northern blots of iNOS and HO-1 mRNA;

FIG. 3 depicts western blots of iNOS and HO-1 protein expression;

FIG. 4 depicts a bar graph of cyclic GMP levels measured by RIA;

FIG. 5 depicts a representative photomicrographs of balloon-injured, perfusion-fixed, elastin-stained left carotid artery (CA) cross-sections of the present invention, wherein panel (A) shows an injured proximal left CA section not exposed to either gel or any other pharmacologic treatment, panel (B) illustrates an injured distal left CA section from the same vessel as shown in (A), except the section was treated immediately after surgery with 1 mg YC-1 of the present invention, and panel (C) shows an injured distal left CA section exposed to empty gel (+DMSO, without YC-1) control; and

FIG. 6 depicts bar graphs of the morphometric analyses for (A) neointimal cross-sectional area, (B) neointimal area/medial wall area ratio, (C) neointimal thickness, and (D) medial wall area.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined and used herein have meanings as commonly understood by a person of ordinary skill in the areas relevant to the present invention. Terms such as “a,” “an,” and “the” are not intended to refer to only a singular entity, but include the general class of which a specific example is used for illustration. The terminology herein is used to describe specific embodiments of the invention, but their usage does not limit the invention, except as outlined in the claims.

All technical and scientific terms used herein have the same meaning as commonly understood by one of ordinary skill in the art to which this invention belongs, unless defined otherwise. All publications mentioned herein are incorporated herein by reference to disclose and describe the methods and/or materials in connection with which the publications are cited.

The arterial remodeling response to endovascular injury is manifested as neointimal formation with a major diminution in luminal patency. While certain characteristics of the adaptive response may be characterized, the regulation of the pathologic process remains largely unknown. Arterial injury has been and continues to contribute to the clinical problem of post-angioplasty stenosis.

Nitric oxide (NO), generated from L-arginine through the action of either constitutive or inducible NO synthase (NOS), is a well established, potent vascular mediator that has been suggested to modulate smooth muscle cell (SMC) phenotype and the arterial response to endovascular injury through cGMP-dependent processes. More recently, carbon monoxide (CO), another diatomic gas, has been shown to serve physiological roles in platelet function, vasomotor tone regulation, and in the control of inflammation and SMC proliferation under inimical conditions.

CO is liberated as a byproduct of the enzymatic action of either inducible (HO-1) or constitutive (HO-2 or HO-3) heme oxygenase on heme that yields free iron and biliverdin. Similar to NO, CO activates soluble guanylyl cyclase (sGC) to stimulate cGMP production, exerting its actions in both autocrine and paracrine fashion.

The potency of CO in stimulating sGC and cGMP production is markedly lower, however, than that of NO. CO stimulation leads to a 4- to 6-fold activation of the purified enzyme, while NO stimulation of the enzyme reaches 400-fold. Despite this relative lack of potency, CO potentially exerts a myriad of vascular cell functions, many of which mimic those of NO. Different responses to regulatory inhibitors and inducers of NOS and HO, as well as significant biochemical differences between the two gases, however, reveal that these systems do indeed represent separate physiological signaling mechanisms.

A physiologically relevant mechanism that sensitizes sGC and cGNM has recently been described, and represents an important source for a new therapeutic intervention. YC-1 [3-(5′-hydroxymethyl-2′-furyl)-1-benzyl indazole] is a chemically synthesized compound that was originally shown to directly stimulate platelet sGC activity and cGMP production and thereby inhibit platelet aggregation and thrombosis. YC-1 also stimulates vascular endothelial and SMC sGC and cGMP, independent of the actions of NO. Furthermore, YC-1 potentiates NO- and CO-induced sGC activation and enhances endothelial NO synthesis and release.

YC-1 is postulated to stabilize the active configuration of sGC and to decrease the dissociation rates of the gases from the activated enzyme. YC-1 stimulates CO-induced sGC with a potency and specific activity similar to that attained with NO stimulation. In addition, YC-1-mediated inhibition of the cGMP-specific phosphodiesterase type 5 (PDE-5) occurs in platelets and in aortic extracts. YC-1 directly activates sGC, to potentiate the stimulatory actions of NO and CO on sGC, and at the same time persistently elevates cGMP (through inhibition of cGMP breakdown).

YC-1, therefore, is a potent activator of the sGC/cGMP system and now foreseen as an attractive new therapeutic agent. The inventors provide that an endogenous or naturally occurring “YC-1-like substance” may be capable of synergizing with endogenous CO and/or NO to stimulate cGMP under pathophysiologic conditions.

The present invention arose from an investigation of the ability of YC-1 to (a) stimulate endogenous vascular cGMP synthesis and (b) attenuate the neointimal response to endovascular injury. For example, investigators found induction of local iNOS and local and systemic HO-1 following balloon injury of rat carotid arteries. Significant acute upregulation of arterial cGMP from YC-1 treatment was also observed, and this was associated with a subsequent and robust diminution in injury-induced neointima formation. Endogenous cGMP represents an important in vivo regulator of cardiovascular function and response and YC-1 serves as a potential new therapeutic strategy in interventional angioplasty.

The established rat carotid artery (CA) provided the model of balloon angioplasty to illustrate the in vivo arterial response to injury. Male Sprague Dawley rats (Harlan, Indianapolis, Ind.) weighing an average 497 g were anesthetized with a combination anesthetic (ketamine, zylazine, and acepromazine; 0.5-0.7 ml/kg, IM; VetMed Drugs, Houston, Tex.), and the left CA and external carotid branch exposed.

A Fogarty 2F embolectomy catheter (Baxter Healthcare Corp., Irvine, Calif.) was introduced into the external carotid branch through an arteriotomy incision and advanced to the aortic arch. The balloon was inflated and withdrawn three times with rotation. The catheter was removed and the external carotid ligated. The overlying tissue was sutured and the skin closed with rodent wound clips.

After full recovery from anesthesia, animals were returned to an animal IP care facility and provided standard rat chow and water ad libitium. Rats were sacrificed by pneumothorax and exsanguinations. Tissues were harvested for use in specific protocols.

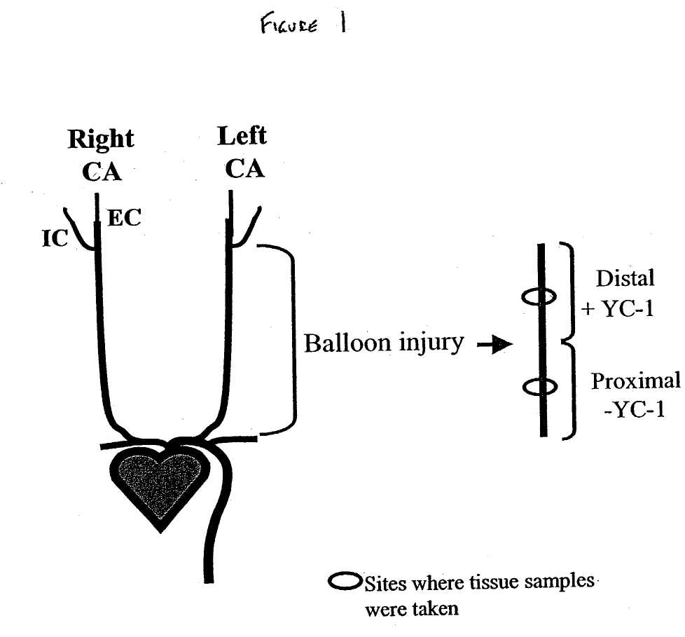

An example of a scheme for YC-1 administration on balloon injured left carotid artery (CA) is shown in FIG. 1. In one embodiment, the entire length of the left CA was balloon injured, immediately followed by perivascular application of YC-1 (1 mg) on the distal section. Sites where tissues were taken for “distal+YC-1” and “proximal−YC-1” are indicated. The right CA served as an uninjured control. EC=external carotid branch; IC=internal carotid branch.

Immediately following balloon injury, at least about 200 μl of a 25% solution of a copolymer gel (Pluronic F-127; BASF Corporation, Mount Olive, N.J.) plus YC-1 (at least about 1 mg in 50 μl DMSO; Calbiochem, La Jolla, Calif.) was administered topically to the exposed adventitia of the distal CA section. Control animals received comparable copolymer gel along (e.g., 200 μl empty gel with 50 μl DMSO) on the distal CA section. In all experiments, the entire length of the left CA was injured (avg. 22 mm), while the contralateral right CA served as an unmanipulated untreated control. Only the distal half of the injured left CA was exposed during the surgery, and this section (approximately 11 mm) was treated with YC-1 (or empty gel) while the proximal half remained untreated. A cohort of animals that was balloon-injured and not exposed to any gel or other treatment served as a separate control group.

Acute studies illustrating enzyme and cGMP induction used freshly obtained tissues that were removed from the sacrificed animal and immediately snap-frozen. In one embodiment, two week studies were performed and morphologic remodeling of the vessel wall was monitored in tissue from animals that were perfusion-fixed transcardially using warmed PBS followed by 10% buffered formalin phosphate. Tissues were fixed, processed by standard procedures, and paraffin-embedded, procedures that are well-known to those of ordinary skill in the art of histology. Five micron sections were cut using a rotary microtome and placed on pre-treated slides. For Verhoff's elastic tissue staining, slides were deparaffinized and stained with Verhoff's solution of alcoholic hematoxylin, ferric chloride, and potassium iodine. Slides differentiated with ferric chloride and counterstained with Van Gieson's solution containing 1% acid fuchsin and picric acid.

Microscopic quantitation of vessel dimensions was performed using Zeiss Image 3.0 (Media Cybernetics) and Adobe Photoshop 4.0™ (Adobe Systems) software systems linked through a CCD color camera (Leaf Microlumina, Leaf Systems, USA) to a Zeiss Axioskop 50 light microscope (Carl Zeiss, Germany). Images were measured for perimeters and areas corresponding to internal and external elastic laminae and lumen. Numerical transformations provided data for neointimal and medial wall areas and vessel diameters.

Cyclic GMP levels were measured in vessel samples with a competitive RIA using ¹²⁵I-labeled cGMP, following instructions from the manufacturer (Amersham Pharmacia Biotech, Piscataway, N.J.). Tissues were freshly removed and immediately snap-frozen. Tissues were homogenized in cold 6% trichloroacetic acid, centrifuged, and the cGMP-containing supernatant removed and ether-washed. The cGMP extract was dried and resuspended. Samples were acetylated, and working standards prepared using serial dilutions for RIA. Separate sections of thoracic aorta incubated in 10 μm sodium introprusside, a potent sGC activator, for 4 minutes at 37° C. provided a positive control for cGMP.

Total tissue RNA was extracted from arteries with guanidium isothiocyanate, and RNA (10 μg) was fractionated on 1% formaldehyde agarose gels and transferred to nitrocellulose membranes. The filters were hybridized with cDNA probes specific for rat HO-1 and iNOS. The cDNA fragments were labeled with [α-³²P]dCTP using a standard random-primed reaction. Membranes were then hybridized with the cDNA probes as described above in Majesky, et al., washed twice, and then exposed to Kodak X-Omat™ film. The membranes were subsequently stripped and rehybridized with a [³²P]-labeled GAPDH probe.

Arterial tissues were homogenized in cold lysis buffer (125 mmole/L Tris-HCl [pH 6.8], 2.5% DTT, 2% SDS, and trace bromophenol blue), boiled, sonicated, and SDS-PAGE was performed on 10% gels for iNOS and 20% gels for HO-1. The blots were electrophoretically transferred to nitrocellulose membranes and blocked for 1 hour.

Membranes were incubated with either a polyclonal antibody specific for macrophage iNOS (1:2000; Transduction Laboratories, Lexington, Ky.) or HO-1 (1:500, Stressgen Biotechnologies Corp., Canada) in PBS for 1 hour at room temperature. After incubation with appropriate secondary antibodies, blots were incubated in enhanced chemiluminescence reagents (Amersham Corp.) and exposed to photographic film.

All data are represented as mean±standard error (SE) of the mean. A two-tailed paired Student's t-test was used to compare same animal data, while all other data were grouped according to treatment and analyzed using an unpaired Student's t-test. P-values <0.05 were considered statistically significant for all comparisons.

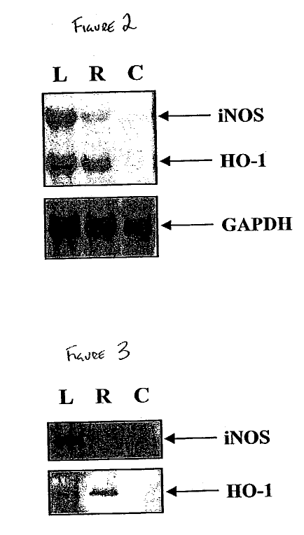

FIG. 2 shows representative results of northern blot analysis of iNOS and HO-1 mRNA expression 24 hours following left CA balloon injury. The lanes represent the ipsilateral injured left CA (L), the contralateral uninjured right CA (R), and CA from control (C) rats which were not subjected to balloon injury. GAPDH is included as a loading control. Between 8 and 10 animals were used per lane to obtain sufficient amounts of RNA. Northern blot analyses demonstrate upregulation of both iNOS and HO-1 mRNA in the ipsilateral left CA (L) 24 hours following balloon injury. The contralateral right CA (R) also demonstrates induced HO-1 mRNA at this time point; however, no detectable iNOS signal is evident in the right CA (R).

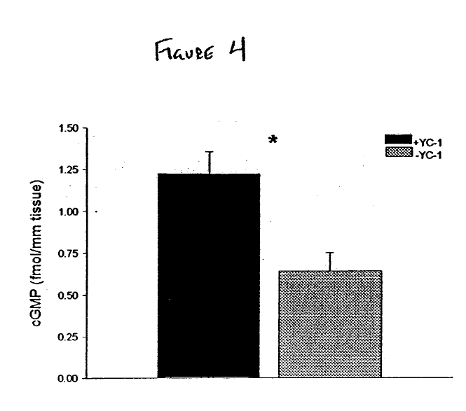

FIG. 3 shows representative western blot analysis of iNOS and HO-1 protein expression 24 hours following left CA (L) balloon injury. The lanes are labeled as in FIG. 2. Western blot analyses similarly reveal induced iNOS and HO-1 protein in the injured left CA (L) 24 hours post-injury. The night CA (R) also shows elevated HO-1 protein and no detectable iNOS protein expression. CA from control (C) rats not subjected to balloon injury do not express iNOS or HO-1 mRNA or protein.

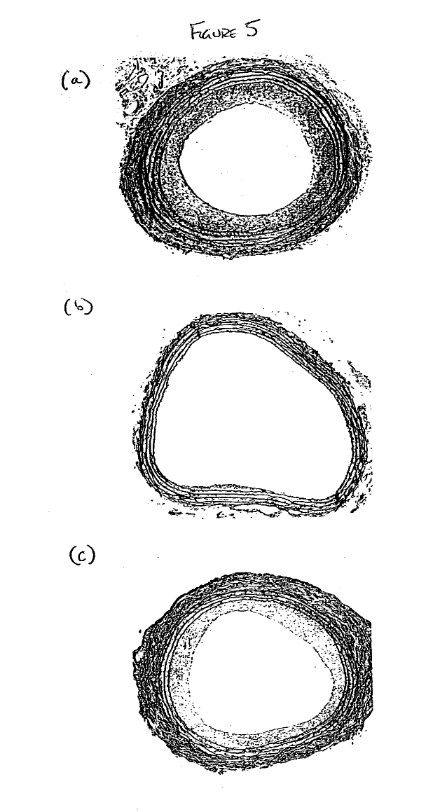

FIG. 4 shows representative cyclic GMP levels measured by RIA in balloon injured left CA sections 24 hours post-injury. Distal arterial sections exposed to YC-1 immediately following injury express a significant (p=0.012, statistically significant with p <0.05) increase in cGMP content compared to the proximal sections from the same vessel not exposed to YC-1. Values represent mean±SE. n=5 animals. The injured CA distal sections exposed to YC-1 exhibit a significant (p=0.012) increase in cGMP content compared to proximal sections (not exposed to YC-1) from the same vessel (1.23±0.13 vs. 0.64±0.11). The CA sections from a separate group of uninjured rats do not show YC-1 induced alterations in cGMP (data not shown). Sections of thoracic aorta were exposed ex vivo to 10 μm sodium nitroprusside, an activator of sGC, for 4 minutes at 37° C. Sodium nitroprusside stimulated approximately a 2-fold increase in vessel wall cGMP levels, similar to that induced by YC-1 in the injured CAs in vivo.

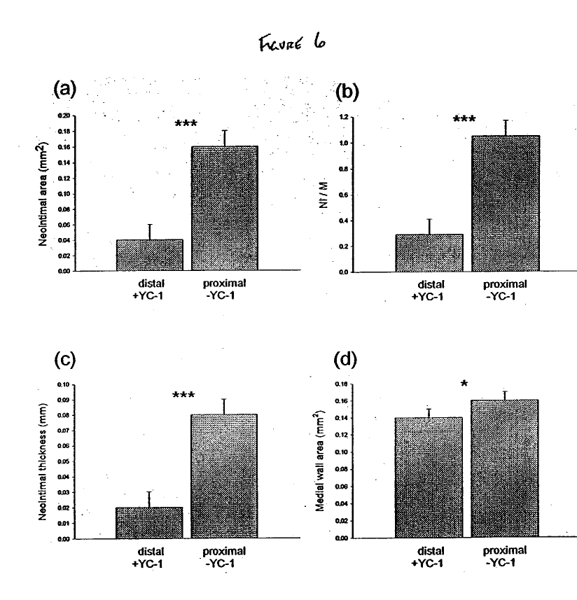

FIG. 5 shows representative photomicrographs of balloon-injured, perfusion-fixed, elastin-stained left CA cross-sections 2 weeks post-injury. Panel (A) shows an injured proximal left CA section not exposed to either gel or any other pharmacologic treatment. A significant and concentric neointima is observed, with elastin fibers of the laminae stained black. Collagen is stained dark red, and is expressed in the abundant adventitia. Panel (B) illustrates an injured distal left CA section from the same vessel as shown in (A), except this section was treated immediately after surgery with 1 mg YC-1. A significantly attenuated neointima is evident, appearing sporadically as a thin layer adjacent to the black internal elastic laminae. Also evident is a decreased adventitia and reduced collagen staining. Panel (C) shows an injured distal left CA section exposed to empty gel (+DMSO, without YC-1) immediately follow surgery. Magnification for all photomicrographs is 100×.

Panel (A) of FIG. 5 shows a Verhoff's elastic tissue-stained cross-section of a balloon-injured left CA. This injured proximal arterial section was not exposed to gel or any other pharmacologic treatment. A significant and concentric neointima is evident, and the elastin-rich laminae are stained black. Collagen is stained dark red, and is highly expressed in the abundant adventitia.

Panel (B) of FIG. 5 illustrates an elastin-stained distal cross-section from the same balloon-injured left CA as shown in FIG. 5(A). The distal section, however, was exposed to YC-1 (1 mg) in gel immediately after surgery. A significantly attenuated neointima is evident, appearing sporadically as a thin layer adjacent to the black internal elastic laminae. Also evident is a decreased adventitia and reduced collagen staining.

FIG. 5(C) illustrates a distal section of a left CA two weeks after injury that had been exposed to empty gel (without YC-1) at time of surgery.

FIG. 6 depicts representative morphometric analyses for (a) neointimal cross-sectional area, (b) neointimal area/medial wall area ratio, (c) neointimal thickness and (d) medial wall area [(a), (b) and (c) statistically significant with p<0.001; (d) statistically significant with p<0.05]. Data for balloon injured left CA distal sections exposed to YC-1 (“distal+YC-1”) and for balloon injured left CA proximal sections not exposed to YC-1 (“proximal−YC-1”) are shown. Values represent mean±SE. For all parameters n=6 animals in the control group and n=9 animals in the YC-1 treated group.

The neointimal cross-sectional area exhibits significant attenuation (−74%; p<0.001) in the distal sections exposed to YC-1 compared to the proximal unexposed sections from the same vessel. Similarly, both the neointimal area/medial wall area ratio (−72%; p=0.001) and the neointimal thickness (-76%; p<0.001) show significant attenuation in the distal YC-1 treated sections compared to the proximal unexposed sections. The medial wall cross-sectional area also shows significant diminution (−12%; p<0.05) in the distal sections exposed to YC-1 compared to the proximal unexposed sections from the same vessel. No major differences were detected between these sections in the control animals for any of these parameters. No differences were detected in the lengths of internal elastic laminae and external elastic laminae between and among all treatment groups.

The results of the experiments illustrate that endogenous cGMP is an important physiologic regulator of the arterial response to injury. Balloon injury was shown to induce both iNOS and HO-1 mRNA transcript and protein expression in the instrumented CA within at least about 24 hours.

Perivascular treatment of the injured CA with YC-1 immediately following surgery significantly increased arterial cGMP levels 24 hours later. YC-1 treatment resulted in a subsequent and robust diminution in injury-induced neointima and vessel wall area after at least about 2 weeks. The results strongly suggest a novel and potentially important therapeutic role for YC-1 in potentiating CO- and/or NO-induced CGMP processes involved in the neointimal response to endovascular injury.

Cyclic GMP is an important intracellular second messenger that is involved in a variety of functional processes. NO binds to the heme moiety of sGC, activating the enzyme to convert cytosolic guanosine triphosphate (GTP) to cGMP. Cyclic GMP mediates its intracellular effects by activation of specific cGMP dependent protein kinases (PKG). Local vascular iNOS is upregulated at both the mRNA and protein levels within 24 hours following balloon injury. This is consistent with previously published reports of medial wall iNOS MRNA and protein upregulation following carotid artery balloon injury (e.g., Yan, Z-Q., Yokota, T., Zhang, W., and Hansson, G. K., Expression of Inducible Nitric Oxide Synthase Inhibits Platelet Adhesion and Restores Blood Flow in the Injured Artery, Circ. Res., 79: 38-44, 1996; and Yan, Z-Q., and Hansson, G. K., Overexpression of Inducible Nitric Oxide Synthase by Neointimal Smooth Muscle Cells, Circ. Res., 82: 21-29, 1998).

In addition to NO, CO provides an alternative physiologic pathway to stimulate cGMP synthesis. The primary source of endogenous CO production is HO-mediated heme degradation, which produces biliverdin and free iron and liberates CO. The marked upregulation of HO-1 by balloon injury indicates that endogenous vascular CO production is highly stimulated following intraluminal injury. Like NO, CO activates sGC to stimulate cGMP production and initiate subsequent cellular functions.

The effects of NO in modulating the arterial response to injury have been recently examined. In rat ileofemoral artery injury, the extent of neointimal thickening was attenuated with application of a NO-releasing agent. eNOS gene transfer resulted in reduction of injury-induced SMC proliferation and neointimal formation in rat CA (Janssens, S., Flaherty, D., Nong, Z., Varenne, O., van Pelt N., Haustemians, C., Zoldhelyi, P., Gerard, R., and Collen, D., Human Endothelial Nitric Oxide Synthase Gene Transfer Inhibits Vascular Smooth Muscle Cell Proliferation and Neoinfima Formation after Balloon Injury in Rats, Circulation, 97: 1272-1281, 1998).

The present inventors have also demonstrated that systemic administration of an HO-1 inducer attenuates the neointimal response to arterial balloon injury. Others have examined the influence of endogenous CO on the adaptive response using in vitro differential gas trapping experiments and suggested CO as the mediator responsible for these protective phenomena but no further examination was performed. The present invention extends such vague observations and provides a novel use of direct YC-1 administration, that is, to induce the manipulation of endogenous cGMP to modify injury-induced vascular remodeling.

The rationale for direct YC-1 administration from the results of FIGS. 2 and 3, where major sources for endogenous CGMTI, NOS-mediated NO and HO-mediated CO are induced by arterial injury. YC-1, as used herein, is a newly synthesized compound that sensitizes the sGC/cGMP system by stabilizing the active configuration of sGC and by decreasing the dissociation rates of diatomic gases from the activated enzyme. YC-1 also has the capacity to inhibit PDE-5 in vascular cells, thereby decreasing cGNP breakdown.

Application of YC-1 on the injured site (e.g., perivascular adminstration on CA) significantly stimulated vessel wall cGMP synthesis. YC-1 stimulated cGMP only in vessels exposed to balloon injury, presumably because of increased release of both iNOS-mediated NO and HO-1-mediated CO. Associated with this cGMP induction, injured arterial sections exposed to YC-1 demonstrated striking diminution in the extent of arterial remodeling after at least about 2 weeks. The protective phenomena were manifested primarily as reduced neointima and diminished luminal stenosis of the injured artery.

The results as described herein show that YC-1 has many potent and protective actions on arterial remodeling. The sGC/cGMP system is markedly potentiated and sensitized to the actions of both CO and NO by YC-1. Cyclic GMP induces the anti-mitogenic cyclindependent kinase (cdk) inhibitor p2i, inhibits cyclin DI expression and cdk4 activation, and delays growth factor-induced cdk2 activation through a transient increase in p27. The actions of cGMP potentially suppress the vascular cell cycle machinery necessary for proliferative neointima formation.

Furthermore, YC-1 prevents collagen-induced mobilization of intracellular Ca 2+ and actin polymerization, thereby decreasing vascular cell migration and possibly inhibiting establishment of a pathologic neointima. Vascular cell apoptosis following balloon angioplasty has been well documented and may provide a necessary regulatory process influencing size and stability of vessel wall lesions. In addition, delayed upregulation of anti-apoptotic genes may enhance cell viability in the newly established vascular lesion.

Since the sGC/cGMP system has been suggested to be involved in NO-induced apoptosis, YC-1 may be capable of altering the extent of vascular cell apoptosis following balloon injury. It is likely that several of these complex molecular mechanisms are involved in the striking protective effect of YC-1 on the adaptive response to vascular injury.

The present invention uses a highly characterized rat carotid artery model of catheter-induced balloon injury to further develop the HO-1 pathway of heme catabolism and its influence on arterial remodeling subsequent to balloon injury. The arterial HO-1 protein was acutely induced from surgery associated with balloon angioplasty. HO-1 (and heme metabolites) may be involved in the arterial response to endovascular intervention.

Pharmacologic administration of hemin upregulated vascular HO-1. Hemin induces the enzyme and provides a heme substrate. Arterial HO-1 induction significantly and consistently decreased the degree of neointima formation following balloon injury (Durante, W., Kroll, M. H., Orloff, G. J., Cunningham, J. M., Scott-Burden, T., Vanhoutte, P. M., and Schafer, A. I., Hemostatic proteins regulate interleukin-I P stimulated inducible nitric oxide synthase expression in cultured vascular smooth muscle cells, Biochem. Pharmacol., 51: 847-853, 1996, incorporated by reference, especially for methods of details).

Acute induction of arterial HO-1 and iNOS mRNA and protein (through Northern and Western blots) was identified from balloon injury. Both of these gases stimulate the sGC/cGMP pathway. YC-1 exerts sGC/cGMP-mediated effects in cells (e.g., cultured cells or tissue). The present invention provides methods of treating a patient to mitigate blood vessel injury and to prevent or reduce the need for restenosis. The treatments of the invention inhibit neointima and the aterial remodeling that occurs after luminal insult to a blood vessel. The methods involve administering to the patient a composition comprising a pharmaceutically effective amount of a soluble guanyl cyclase activator, such as YC-1. The soluble guanyl cyclase activator of the present invention may also be an endogenous or naturally occurring substance that accomplishes the same physiological function of YC-1.

Another method of the invention involves forming a therapeutic composition having a pharmaceutically effective amount of a soluble guanyl cyclase activator in or on a biologically acceptable carrier and administering the composition on or near the locus of the trauma so that neointima formation on or near the locus of the trauma is reduced. The soluble guanyl cyclase activator may be YC-1 and the carrier may be a catheter, such as a balloon angioplasty catheter or an embolectomy catheter. Additionally, the carrier may be a gel such as a copolymer gel.

In other embodiments, the catheter may be coated with a pharmaceutically effective amount of the soluble guanyl cyclase activator. The soluble guanyl cyclase activator may be administered during withdrawal of the balloon angioplasty catheter. Another example provides a catheter fitted with an applicator for delivery of the soluble guanyl cyclase activator.

Still further embodiments of the present invention include a stent coated with YC-1 or other suitable endogenous or naturally occurring sGC inducer. Such a coated stent inhibits blood vessel remodeling that would other require the surgical removal and restenosis of, for example, a typical uncoated stent.

The coated stent of the resent invention may have biodegradable materials such as poly(L-lactic acid) (PLA), poly(glycolic acid), poly(L-lactic-co-glycolic acid) (PGA), poly(e-caprolactone) (PLGA), polyortho esters, polyanhydrides or combinations thereof. The biodegradable materials may be layered.

The size and characteristics of the degradable polymers for use with the present invention may be varied depending on the size and structural requirements of the target blood vessel. Either synthetic or naturally occurring polymers may be used, and while not limited to this group, some types of polymers that might be used, in addition to those indicated above, are polysaccharides (e.g. dextran, ficoll), proteins (e.g. poly-lysine), poly(ethylene glycol), or poly(methacrylates). Different polymers, because of their different size and shape, will produce different diffusion characteristics for YC-1 in the target tissue or organ.

Hydrolytically labile bonds between the polymer and YC-1, if desired, may be varied to provide a controlled-release matrix. The rate of hydrolytic degradation, and thus of YC-1 release, can be altered from minutes to months by altering the physico-chemical properties of the bonds between the YC-1 and the polymer. The rate of release may be affected by, e.g., the nature of the bond, e.g., ionic, thioester, anhydride, ester, and amide links, in order of decreasing lability; stereochemical control, building in varying amounts of steric hindrance around the bonds which are to be hydrolyzed; electronic control, building in varying electron donating/accepting groups around the reactive bond, controlling reactivity by induction/resonance; varying the hydrophilicity/hydrophobicity of spacer groups between the YC-1 and the polymer; varying the length of the spacer groups, increasing the length of the bond to be hydrolyzed so that the bond is more accessible to water; and using bonds susceptible to attack by enzymes present in the extracellular matrix or on the surface of cells.

The properties of the controlled release matrix can be varied, according to methods described in by, e.g, Saltzman, et al., 1991, Chemical Engineering Science, 46:2429-2444; Powell, et al., 1990, Brain Research, 515:309-311; Dang, et al., 1992, Biotechnology Progress, 8:527-532; Saltzman, et al., 1989, Biophysical Journal, 55:163-171; Radomsky, et al., 1992, Biology of Reproduction, 47:133-140; Saltzman, et al., 1992, Journal of Applied Polymer Science, 48:1493-1500; Sherwood, et al., 1992, Bio/Technology, 10: 1446-1449, to vary the rate of polymeric YC-1 conjugate release into the tissue.

Among the variables that affect conjugate release kinetics are: controlled release polymer composition, mass fraction of YC-1-polymer conjugate within the matrix (increasing mass fraction increases release rate), particle size of YC-1-polymer conjugate within the matrix (increasing particle size increases release rate), composition of polymeric YC-1 conjugate particles (which can be varied by adding free drug agents or inert agents that influence particle solubility), and size (increasing surface area increasing the release rate), and shape (changing the pattern, e.g., first order, zeroth order, etc.) of the controlled release matrix. Suitable polymers for use as controlled-release matrices include poly(ethylene-co-vinyl acetate), poly(DL-lactide), poly(glycolide), copolymers of lactide and glycolide, and polyanhydride copolymers.

In addition to coated stents as just described, other embodiments of the present invention include a balloon angioplasty catheter coated with YC-1 or a catheter fitted with an applicator for applying YC-1 during withdrawal of the catheter.

Still further embodiments include a kit with a catheter adapted for administration of YC-1 to a blood vessel, a pharmaceutically effective amount of YC-1 adapted for administration by the catheter and instructions for administering the YC-1 with the catheter. The kit may include instructions printed on paper. The instructions, however, may be on a videotape or CD-ROM that may include, for example, analog or digital, respectively, video demonstrations instructing on the use of the kit.

While the making and using of various embodiments of the present invention are discussed in detail below, it should be appreciated that the present invention provides many applicable inventive concepts which can be embodied in a wide variety of specific contexts. The specific embodiments discussed herein are merely illustrative of specific ways to make and use the invention and do not delimit the scope of the invention. Various modifications and combinations of the illustrative embodiments, as well as other embodiments of the invention, will be apparent to persons skilled in the art upon reference to the description.

What is claimed is:

1. A method of treating a patient to mitigate blood vessel injury caused by angioplasty performed on the patient, the method comprising administering to the patient a composition comprising a pharmaceutically effective amount of a soluble guanyl cyclase activator.

2. The method of claim 1, wherein the soluble guanyl cyclase activator comprises YC-1.

3. A stent coated with YC-1.

4. The stent of claim 3, wherein the stent comprises biodegradable materials.

5. The stent of claim 4, wherein the biodegradable materials comprise poly(L-lactic acid) (PLA), poly(glycolic acid), poly(L-lactic-co-glycolic acid) (PGA), poly(e-caprolactone) (PLGA), polyortho esters, polyanhydrides or combinations thereof.

6. The stent of claim 4, wherein the biodegradable materials are layered.

7. A balloon angioplasty catheter coated with YC-1.

8. The balloon angioplasty catheter of claim 7, wherein the catheter comprises an applicator for application of YC-1 during withdrawal of the catheter.

9. A kit comprising:
a catheter adapted for administration of YC-1 to a blood vessel;
a pharmaceutically effective amount of YC-1 adapted for administration by the catheter; and
instructions for administering the YC-1 with the catheter.

10. The kit of claim 9, wherein the instructions are printed on paper.

11. A method of treating arterial trauma, the method comprising:
forming a therapeutic composition comprising a pharmaceutically effective amount of a soluble guanyl cyclase activator in or on a biologically acceptable carrier; and
administering the composition on or near the locus of the trauma, whereby neointima formation on or near the locus of the trauma is reduced.

12. The method of claim 11, wherein the soluble guanyl cyclase activator comprises YC-1.

13. The method of claim 11, wherein the carrier comprises a catheter.

14. The method of claim 13, wherein the catheter is a balloon angioplasty catheter.

15. The method of claim 13, wherein the catheter is an embolectomy catheter.

16. The method of claim 13, wherein the catheter is coated with a pharmaceutically effective amount of the soluble guanyl cyclase activator.

17. The method of claim 11, wherein the carrier is a ballon angioplasty catheter and wherein the soluble guanyl cyclase activator is administered during withdrawal of the balloon angioplasty catheter.

18. The method of claim 11, wherein the carrier comprises a catheter fitted with an applicator for delivery of the soluble guanyl cyclase activator.

19. The method of claim 18, wherein the soluble guanyl cyclase activator comprises YC-1.

20. The method of claim 11, wherein the carrier comprises a gel.
